SEQUENCE LISTING (6 sequences)

SEQ ID NO: 1 - DNA, 1548 bp, Formosa agariphila
  CDS (1)..(1548)
atgctagaaaaaactactcttaaaaatattattttaatacactttctaatgtttttagctgtagtaacagctcaaaccgcgcccgatgaagatacatctgctattacaagatgtactgctgaaggtacaaaccctgtaagagaaacagatattccaaaccccgtaaatgtgggaactatcgatgaccgtagttgttatgctaattataaggagagtacagtttatggaaaaacttggggtgtctataacataacgtttgattctaatgattttgatacctctttacaaccaagaatagaacgttcattaagccgctccagcgaaacgggtattggaagttatgctaggttaacaggagtttttagaattcttgaagttggagatacttcagggaccagtcaggatggaacttatcttgctcaagcaaaaggtaagcacacagggggcggtggttcaccagatccagcaatttgtttgtatttagcaaaacctgtttatggtacaggggaagatgccgataaacaagtctcgttcgatatttatgcagagcgtattttatatcgaggtggagaaggagatggaagagaaattgttttcttaaagaatgtaaagaaggatgaagaaactaattttgagttagaagttggtttcaaagaagatcctaacgatgtctctaaaaaaatacagtattgtaatgctgtcataggaggagatacttttaattggaatattccagaaccagaaagaggaacagaatcgggtattagatatggagcatatagagttaaaggaggacgagctcaaataaggtgggccaataccacctaccaaaaggtagaaaacgtagaggttacaaatccaggaccaataggagatgtttataaattaaaaaatgttgctacaggtcagtatttatcagattcgggagttagtgcttcagcagtaattatgagtgattcaggtgaagcgcaaaataattattggacttttgtagaaagcgggagtttgtttaacatagataatgaaacttttggcatactgcgtgcaccaggtgcaggtggtccaggaggtgcttatgtggtagtaagtacaacaaaagaaggtccttcttctgatggcgataaggtttggacgatacattataatgaatctaacgatacatatcgatttgaatcgggtagtagtggcagatttatgtatcaagaaataaatggaaatgttactcatatctccgctatgaataccgatgatagaagtgtttggaaagctattgctgttgagtctctgagtgtagatgagaacgcgattttagcttctgatgtacgtgtatttcctaacccagcaagcgatagttttacgatttccttaaaaacaataaatcatgttacagttaatatatacgatgttttaggaaatactatttttaaaagtgaatttaatggggatactatacaaataagaaataaagggcaattcaaagctggggtttatcttatacagttaactgataaaaataacaacaagtatcataagaaattgatagtaaaa

SEQ ID NO: 2 - protein, 516 aa, Formosa agariphila
MLEKTTLKNIILIHFLMFLAVVTAQTAPDEDTSAITRCTAEGTNPVRETDIPNPVNVGTIDDRSCYANYKESTVYGKTWGVYNITFDSNDFDTSLQPRIERSLSRSSETGIGSYARLTGVFRILEVGDTSGTSQDGTYLAQAKGKHTGGGGSPDPAICLYLAKPVYGTGEDADKQVSFDIYAERILYRGGEGDGREIVFLKNVKKDEETNFELEVGFKEDPNDVSKKIQYCNAVIGGDTFNWNIPEPERGTESGIRYGAYRVKGGRAQIRWANTTYQKVENVEVTNPGPIGDVYKLKNVATGQYLSDSGVSASAVIMSDSGEAQNNYWTFVESGSLFNIDNETFGILRAPGAGGPGGAYVVVSTTKEGPSSDGDKVWTIHYNESNDTYRFESGSSGRFMYQEINGNVTHISAMNTDDRSVWKAIAVESLSVDENAILASDVRVFPNPASDSFTISLKTINHVTVNIYDVLGNTIFKSEFNGDTIQIRNKGQFKAGVYLIQLTDKNNNKYHKKLIVK

SEQ ID NO: 3 - DNA, 783 bp, Formosa agariphila
  CDS (1)..(783)
caaaccgcgcccgatgaagatacatctgctattacaagatgtactgctgaaggtacaaaccctgtaagagaaacagatattccaaaccccgtaaatgtgggaactatcgatgaccgtagttgttatgctaattataaggagagtacagtttatggaaaaacttggggtgtctataacataacgtttgattctaatgattttgatacctctttacaaccaagaatagaacgttcattaagccgctccagcgaaacgggtattggaagttatgctaggttaacaggagtttttagaattcttgaagttggagatacttcagggaccagtcaggatggaacttatcttgctcaagcaaaaggtaagcacacagggggcggtggttcaccagatccagcaatttgtttgtatttagcaaaacctgtttatggtacaggggaagatgccgataaacaagtctcgttcgatatttatgcagagcgtattttatatcgaggtggagaaggagatggaagagaaattgttttcttaaagaatgtaaagaaggatgaagaaactaattttgagttagaagttggtttcaaagaagatcctaacgatgtctctaaaaaaatacagtattgtaatgctgtcataggaggagatacttttaattggaatattccagaaccagaaagaggaacagaatcgggtattagatatggagcatatagagttaaaggaggacgagctcaaataaggtgggccaataccacctaccaaaaggtagaaaacgtagaggttaca

SEQ ID NO: 4 - protein, 261 aa, Formosa agariphila
QTAPDEDTSAITRCTAEGTNPVRETDIPNPVNVGTIDDRSCYANYKESTVYGKTWGVYNITFDSNDFDTSLQPRIERSLSRSSETGIGSYARLTGVFRILEVGDTSGTSQDGTYLAQAKGKHTGGGGSPDPAICLYLAKPVYGTGEDADKQVSFDIYAERILYRGGEGDGREIVFLKNVKKDEETNFELEVGFKEDPNDVSKKIQYCNAVIGGDTFNWNIPEPERGTESGIRYGAYRVKGGRAQIRWANTTYQKVENVEVT

SEQ ID NO: 5 - protein, 273 aa, Formosa agariphila
  misc_feature (1)..(273): protein sequence of the catalytic module of the ulvan lysae of F. agariphila DSM 15362 produced in recombinant form
MGSSHHHHHHGSQTAPDEDTSAITRCTAEGTNPVRETDIPNPVNVGTIDDRSCYANYKESTVYGKTWGVYNITFDSNDFDTSLQPRIERSLSRSSETGIGSYARLTGVFRILEVGDTSGTSQDGTYLAQAKGKHTGGGGSPDPAICLYLAKPVYGTGEDADKQVSFDIYAERILYRGGEGDGREIVFLKNVKKDEETNFELEVGFKEDPNDVSKKIQYCNAVIGGDTFNWNIPEPERGTESGIRYGAYRVKGGRAQIRWANTTYQKVENVEVT

SEQ ID NO: 6 - protein, 47 aa, Artificial Sequence
  Signal sequence
MVFFKDLFIFKSLIKGSLYSGHMKKKLLNYLPLFALMLFTVSMMAQT

(19) United States
(12) Patent Application Publication
(10) Pub. No.: US 2019/0323048 A1
(43) Pub. Date: Oct. 24, 2019

(54) NOVEL ULVAN LYASE AND USE THEREOF FOR CLEAVING POLYSACCHARIDES

(71) Applicants: Centre National de la Recherche Scientifique, Paris (FR); Sorbonne Université, Paris (FR)

(72) Inventors: Gurvan MICHEL, Plougoulm (FR); Alexia GUILLOUZO, Champhol (FR); Bertille MASSÉ, Guipavas (FR); Sabine GÉNICOT, Saint-Pol-de-Léon (FR); Agnès GROISILLIER, Sibiril (FR)

(21) Appl. No.: 16/333,207

(22) PCT Filed: Sep. 12, 2017
(86) PCT No.: PCT/FR2017/052414

§ 371 Date: Mar. 13, 2019

(30) Foreign Application Priority Data
Sep. 13, 2016 (FR) 1658505

(51) Int. Cl. C12P 19/12; C12N 9/88

(52) U.S. Cl. CPC C12P 19/12; C12Y 402/02; C12N 9/88

(57) ABSTRACT

Some embodiments are directed to a novel ulvan lyase, a nucleic acid sequence coding for said enzyme, a vector comprising said coding sequence, a method for manufacturing said ulvan lyase, and a method for producing ulvan oligosaccharides with biological activity using said enzyme

### CROSS REFERENCE TO RELATED APPLICATIONS

This application is a national phase filing under 35 C.F.R. § 371 of and claims priority to International Patent Application No. PCT/FR2017/052414, filed on Sep. 12, 2017, which claims the priority benefit under 35 U.S.C. § 119 of French Application No. 1658505, filed on Sep. 13, 2016, the contents of which are hereby incorporated in their entireties by reference.

### BACKGROUND

The presently disclosed subject matter relates to a novel ulvan lyase, to a nucleic acid sequence coding for that enzyme, to a vector comprising the coding sequence, to a process for manufacturing that ulvan lyase, and to a process for producing ulvan oligosaccharides with biological activity utilizing that enzyme.

The presently disclosed subject matter finds its applications mainly in agriculture, the animal and human nutrition industries, cosmetics, pharmacy, etc. . . . but also in the field of laboratory research for the production of standard molecules, size markers, etc. . . . or for the fine analysis of the structure of compositions of ulvan polysaccharides of varied nature and origin.

In the description below, the references in square brackets ([ ]) refer to the list of references presented at the end of the text.

The green macroalgae of the family of the Ulvaceae are present everywhere on the Earth and are very commonly encountered on the coasts. These algae are often involved in the algal proliferation favored by the eutrophication of coastal waters giving rise to the “green tides”. However, until the present time, they constitute an enormous biomass under-exploited in biotechnology, and utilized essentially as compost.

The major complex anionic polysaccharides present in the walls of the ulvae, the ulvans, are uronic and sulfated polysaccharides possessing original structures and representing a source of biopolymers whose functionalities are as yet little explored.

The ulvans and the derived oligosaccharides have numerous interesting biological properties, in particular due to their resemblance to the sulfated glycosaminoglycans of animals (e.g. heparins, dermatan sulfates, etc. . . . ). The ulvans are made up of different disaccharide repeating units built up of L-rhamnose units, D-glucuronic acids, L-iduronic acids, D-xyloses and ester-sulfate groups. The two principal repeating motifs are called aldobiuronic acid or ulvanobiuronic acids, or respectively A (a_{3S}) and B (B_{3S}), the formulae whereof are as follows:

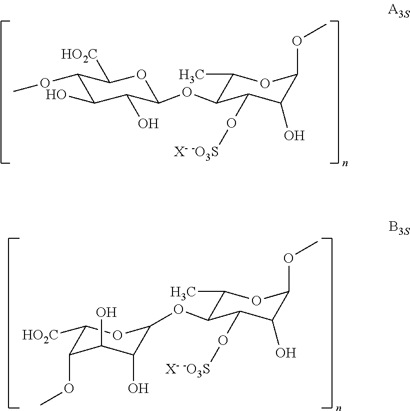

Motif A (A_{3S}) is beta-D-1,4-glucuronic acid (1→4) alpha-L-1,4-rhamnose 3-sulfate. Motif B (B3S) is alpha-L-1,4-iduronic acid (1→4) alpha-L-1,4-rhamnose 3-sulfate. It is noted that L-iduronic acid is the C5 epimer of D-glucuronic acid. The uronic acids are sometimes replaced by xylose residues sulfated at O-2.

The ulvans possess unique physical and chemical properties which make them attractive candidates for novel agri-food, pharmaceutical and cosmetic applications. The ulvans possess very original structures made up of rare sugars or monosaccharides such as rhamnose and iduronic acid. Rhamnose is a compound of the surface antigens of many microorganisms specifically recognized by the lectins of mammals. It is also used for the synthesis of flavorings. Iduronic acid is used for the synthesis of the glycosaminoglycans, for example heparin. Thus, the ulvans can be considered as biomimetics of the glycosaminoglycans of animals and man, by reason of the joint presence of sulfated sugars and of D-glucuronic and L-iduronic acids.

Apart from the monomers, the ulvans and oligo-ulvans exhibit interesting biological properties. In fact, studies have shown, for example, that the oligo-ulvans have antitumor, antiviral, in particular anti-influenza, and anticoagulant activities. A non-exhaustive list of potential applications of the ulvans has been proposed by Lahaye and Robic (Biomacromolecules, 8: 1765-1774, 2007) [1].

In this context, a better understanding of the structure of the ulvans and the development of processes making it possible to fragment the ulvans in oligomeric or monomeric form assumes very great interest.

Currently in the absence of making it possible to know the ulvans better and to degrade them more efficiently, the algae, in particular the green algae, are essentially composted and little industrial development of these is performed. This is all the more regrettable since the source is abundant and sometimes a nuisance in terms of pollution of our sea coasts. Their elimination is currently effected by the compost route.

Acidic hydrolysis is essentially the method utilized to liberate the oligosaccharides of ulvans, but this method is random and alters the structure of the polymer.

Certain marine bacteria secrete enzymes involved in the specific degradation of algal polysaccharides. Utilization of a specific enzyme makes it possible to liberate oligosaccharides reproducibly while preserving their structure and biological activity. An ulvan lyase from Nonlabens ulvanivorans PLR (previously named Persicivirga ulvanivorans PLR) has been described and protected by patent (Nyvall Collen et al., J. Biol. Chem., 286(49): 42063-42071, 2011; International application WO 2011157966) [2,3]. However, this enzyme is extremely weakly expressed in a soluble and active form in Escherichia coli [2] (see also our own result below, FIG. 1); which in practice prevents any industrial exploitation of that enzyme.

There is therefore a real need for a process remedying the defects, disadvantages and obstacles of the related art, in particular for a process making it possible to degrade the ulvans in order to develop this bio-resource, derived in particular from the green algae and thus to liberate in a controlled and reproducible manner novel ulvan oligosaccharides of defined structures, and this while preserving the fine structure and thus the biological properties of the oligosaccharides with regard to cosmetic, agri-food and medical applications.

### SUMMARY

Some embodiments addresses or enhance the defects, disadvantages and obstacles discussed above by identifying, cloning and heterologously overexpressing in Escherichia coli and purifying, for the first time, a novel active ulvan lyase, from the complete genome of the marine bacterium Formosa agariphila DSM 15362 (Mann et al., Appl. Env. Microbiol., 79: 6813-6822, 2013; Nedashkovskaya et al., Int. J. Syst. Evol. Microbiol., 56: 161-167, 2006) [4, 5]. Moreover, the utilization of this enzyme for the complete degradation of ulvan made it possible to isolate and to purify novel ulvan oligosaccharides (or oligo-ulvans) having a unique, sulfated chemical structure. These sulfated oligosaccharides are potentially interesting for applications in biomedical science, cosmetics, nutrition, elicitation of plants, etc. . . . . This enzyme also makes it possible to increase the extraction yield of other compounds (e.g. proteins, pigments, secondary metabolites) from green macroalgae containing ulvan. To this end, some embodiments demonstrate that the ulvan lyase of Formosa agariphila DSM 15 362 makes it possible to: (i) depolymerize ulvan (major polysaccharide of the green macroalgae), liberating oligosaccharides with biological activity while preserving their native structure; (ii) directly degrade green macroalgae to generate fractions containing oligosaccharides with biological activity; and (iii) increase the extraction yield of other compounds (e.g. proteins, pigments, etc. . . . ) from green macroalgae.

The presently disclosed subject matter thus relates to the ulvan lyase of Formosa agariphila DSM 15362 which can be included in the amino acid sequence SEQ ID No: 2 or to an ulvan lyase that can be included in an amino acid sequence having at least 53% sequence identity with the sequence SEQ ID No: 2, or to an ulvan lyase from Formosa agariphila DSM 15362 that can be included in the amino acid sequence SEQ ID No: 4 or 5 or to an ulvan lyase that can be included in an amino acid sequence having at least 67% sequence identity with the sequence SEQ ID No: 4. The sequences SEQ ID Nos: 4 and 5 represent, respectively, the catalytic module of the ulvan lyase of Formosa agariphila DSM 15362 and the module produced in recombinant form (in the cytoplasm of E. coli. The additional N-terminal sequence is a polyhistidine tag to facilitate purification). Possibly, the presently disclosed subject matter relates to the catalytic module of the ulvan lyase of Formosa agariphila DSM 15362 that can be included in the sequence SEQ ID No: 4, or in the sequence SEQ ID No: 5.

According to the presently disclosed subject matter, these ulvan lyases or ulvan lyase catalytic modules, whatever may be their sequence, can further include, and their N-terminal end, a signal sequence or addressing sequence. This signal sequence can be one of the signal sequences known to those of ordinary skill in the art such that the protein, when it is synthesized in a host cell, is directed towards an organelle or a particular zone of the host cell. It can for example be a signal sequence found in the sites specializing in the prediction of signal peptides, for example http://www.cbs.dtu.dk/services/SignalP/[10]. It can for example be the sequence SEQ ID No: 6, for example at the N-terminus of the sequence SEQ ID No: 4, of the appended list of sequences. This signal sequence can be cleaved after synthesis of the protein or not. The cleavage processes known to those of ordinary skill in the art can be utilized, for example those involving proteases specific to a cleavage site. A signal sequence exhibiting such a site is then chosen.

All these amino acid sequences have never been described in the related art as being active for degrading the ulvans by depolymerization according to the presently disclosed subject matter. The presently disclosed subject matter also relates to the utilization of an amino acid sequence as defined above for the specific and complete degradation of ulvan to produce ulvan oligosaccharides (or oligo-ulvans).

The presently disclosed subject matter also relates to a nucleic acid coding for an ulvan lyase according to some embodiments. It can for example be a nucleic acid included in the sequence SEQ ID No: 1, or a nucleic included in a sequence having at least 53% identity with the sequence SEQ ID No: 1. It can for example be a nucleic acid included in the sequence SEQ ID No: 3, or in a sequence having at least 67% identity with the sequence SEQ ID No: 3.

The presently disclosed subject matter also relates to a vector comprising a nucleic acid according to some embodiments. The vector can be one of the vectors known to those of ordinary skill in the art for manufacturing proteins by genetic recombination, for example an expression vector. In general it is in particular selected depending on the host cell chosen.

The presently disclosed subject matter also relates to a host cell comprising a nucleic acid sequence according to some embodiments, or a vector according to some other embodiments. The host cell or cellular host can be any host appropriate for the production of the ulvan lyase of the presently disclosed subject matter from the nucleic acid or the vector of some embodiments. It can for example be E. coli, Pischia pastoris, Saccharomyces cerevisiae, cells of insects, for example an insect cells-baculovirus system (for example SF9 insect cells utilizing a baculovirus expression system), or of mammals.

The presently disclosed subject matter also relates to a process for production of an ulvan lyase according to some embodiments by genetic recombination utilizing a nucleic acid according to some embodiments, or a vector according to some embodiments. The processes of genetic recombination known to those of ordinary skill in the art are utilizable. The marine or terrestrial origin does not influence the possibility of heterologous recombination and expression. The ulvan lyase of the presently disclosed subject matter can also be produced by culturing the microorganism F. agariphila in a medium enabling the growth of the marine microorganism. It can for example be a ZoBell liquid culture medium. The culturing pH possibly lies between 7.5 and 8.4, possibly at pH 8.0. The culturing temperature could lie between 4 and 33° C., possibly at 22° C. The culturing may be performed with a concentration of NaCl of 10 to 80 g/I, possibly 35 g/I [5]. These processes for production of the ulvan lyase of some embodiments can further include a stage of recovery of the ulvan lyase. This recovery or isolation stage can be performed by techniques known to those of ordinary skill in the art. It can for example be a technique selected from an electrophoresis, a molecular sieving, an ultracentrifugation, a differential precipitation, for example with ammonium sulfate, by ultrafiltration, a membrane or gel filtration, an ion exchange, an elution over hydroxyapatite, a separation by hydrophobic interactions, or any other known techniques.

The microorganism F. agariphila or any host cell transformed for a manufacture by genetic recombination according to some embodiments, can also be utilized directly for degrading ulvans, in their natural environment or in culture. The conditions enabling the degradation of the ulvans, when a host cell or microorganism is used, are, in culture, a discontinuous or continuous system, for example a culture reactor containing a culture medium appropriate to the development of the microorganism.

The presently disclosed subject matter also relates to a process for degradation of ulvans comprising a stage of contacting ulvans with an ulvan lyase according to some embodiments, or with a host cell according to some embodiments, under conditions enabling the degradation of the ulvans by enzymatic digestion by the ulvan lyase or the host cell or the microorganism F. agariphila.

For the enzymatic digestion, the determination of the Michaelis Menten constants (Km and Vmax) easily enables those of ordinary skill in the art to find the optimal conditions of concentration of the ulvan lyase utilized and of concentration of the ulvans for the degradation of the ulvans in the medium where they are situated or in the medium into which they have been placed. The pH may also lie between 6.0 and 8.5, possibly at 7.5. This is in fact the optimal pH range. The temperature (optimal) possibly lies between 30 and 40° C. The ionic strength can possibly lie between 0 and 300 mM NaCl, possibly at 30 mM NaCl.

The presently disclosed subject matter advantageously makes it possible to mobilize the very great currently unexploited resource of algae, in particular green algae. Some embodiments further makes it possible to promote the biodegradation of algae, in particular green algae, to produce original molecules which are fragments of ulvans or oligo-ulvans, for example oligosaccharides, for example also hydrocolloids, and to offer a novel source of rare monosaccharides for cosmetic and agri-food applications and medicaments or pharmaceutical and parapharmaceutical formulations.

The degradation products of the ulvans give access to novel products which can be food, cosmetic, pharmaceutical and parapharmaceutical active substances utilizable in the agri-food, cosmetic, pharmaceutical and parapharmaceutical fields. These novel products can also be non-active products which however exhibit a neutrality and/or stability which is very interesting for use in each of these fields.

The utilization of the ulvan lyase of some embodiments further gives access to rare monosaccharides utilizable as synthons in glycochemistry. The degradation of ulvans by the ulvan lyase of the presently disclosed subject matter combined with other enzymes can give access to iduronic acid (rare sugar) utilized for the synthesis of synthetic glycosaminoglycans.

The presently disclosed subject matter also opens up novel prospects for utilization of these algae for applications in bioenergy and in chemistry. The production of oligosaccharide fragments can give base molecules for the production of other molecules. The depolymerization of ulvan should facilitate its fermentation by microorganisms leading to the production of methane for example.

Other characteristics and advantages will further appear to those of ordinary skill in the art in the reading of the examples below, given by way of illustration, and not limiting, with reference to the appended diagrams.

### BRIEF DESCRIPTION OF THE FIGURES

FIG. 1 shows the comparison of the expression in E. coli BI2(DE3) of the catalytic module of the ulvan lyase of N. ulvanivorans PLR (A and B) and F. agariphila DSM 15362 (C and D). Plates A and C correspond to SDS-PAGE analyses. Plates B and D correspond to Western blot analyses with an anti-His-Tag antibody. FW=Flow-through of the nickel affinity chromatography; E1 and E2: eluates 1 and 2 of the nickel affinity chromatography.

FIG. 2 shows an activity test of the recombinant ulvan lyase BN863_22190_cat as a function of the NaCl concentration (A), of the nature of the buffer (B) and of the pH (for the best buffer, C). The activity was followed by spectrophotometry at 235 nm.

FIG. 3 shows a FACE analysis (Fluorophore-assisted Carbohydrate Electrophoresis) of the oligo-ulvans liberated by extensive digestion of ulvan by the recombinant ulvan lyase BN863_22190_cat. These samples are named FracX where X is a fraction number corresponding to the purification of these oligosaccharides by size exclusion chromatography. The oligosaccharides DP2 and DP4 are standard oligo-ulvans liberated by the wild ulvan lyase of Nonlabens ulvanivorans PLR [1].

FIG. 4 shows a mass spectrum of the fraction 48 recorded on the BIBS platform (INRA, Nantes). The mass/charge (m/z) main peaks correspond to a disaccharide Δ-R3S.

FIG. 5 represents a mass spectrum of the fraction 39 recorded on the BIBS platform (INRA, Nantes). The mass/charge (m/z) main peaks correspond to a tetrasaccharide Δ-R3S-Xyl-R3S.

### EXAMPLES

### Example 1: Expression of the Ulvan Lyase of Formosa agariphila

The gene coding for that ulvan lyase (BN863_22190) was identified in the genome of F. agariphila DSM 15362 by sequence homology with the ulvan lyase of Nonlabens ulvanivorans PLR (formerly Persicivirga ulvanivorans) [2, 3].

The protein encoded by this gene has a molecular mass of 56,630 Da (516 residues). It has a modular architecture with an N-terminal signal peptide followed by a catalytic module, a module of unknown function and a C-terminal type IX secretion module. Over the totality of its sequence, this protein has 52% sequence identity with that of the ulvan lyase of N. ulvanivorans PLR, as determined by sequence alignment by the CLUSTALW software. The catalytic module of the ulvan lyase of F. agariphila DSM 15362 (residues 25-285, 28,738 Da) exhibits 66% sequence identity with the sequence of the catalytic module of the ulvan lyase of N. ulvanivorans (residues 24-291).

The nucleotide sequence corresponding to the catalytic module of the ulvan lyase of F. agariphila DSM 15362 was cloned into the vector pFO4 (derived from the commercial vector pET15b, [6]), and the corresponding recombinant protein was overexpressed in Escherichia coli (SEQ ID No: 5). For comparison, a similar study was performed for the catalytic module of the ulvan lyase of N. ulvanivorans PLR.

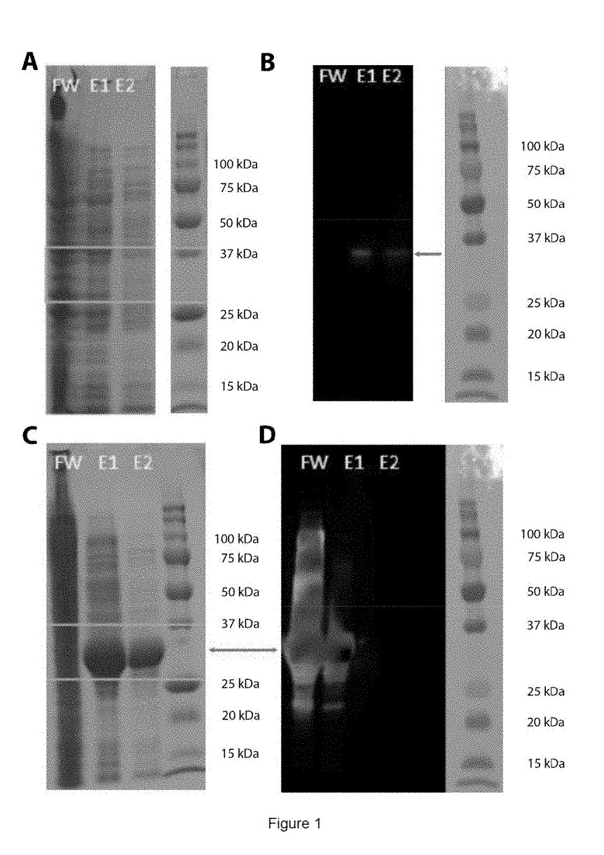

The results presented in FIG. 1 show that the catalytic module of the ulvan lyase of N. ulvanivorans PLR is not visible by electrophoreses on denaturing polyacrylamide gel stained with Coomassie blue (FIG. 1A), even after purification by nickel affinity chromatography. This enzyme was nonetheless weakly detected by an anti-Histidine-TAG antibody (FIG. 1B, see arrow); which confirmed very weak soluble expression of this protein (a few μg per liter of culture). In contrast, the catalytic module of the ulvan lyase of F. agariphila DSM 15362 (referred to below as BN863_22190_cat) is very strongly expressed in soluble form, as indicated by the wide band visible at −29 kDa in the SDS-PAGE (FIG. 1C). This was confirmed by Western blot (FIG. 1D), which is a much more sensitive technique than SDS-PAGE. The production yield of this recombinant protein was 30 mg per liter of culture, that is to say at least 10,000 times more than the recombinant ulvan lyase of N. ulvanivorans PLR.

### Example 2: Biological Activity of the Ulvan Lyase of Formosa agariphila

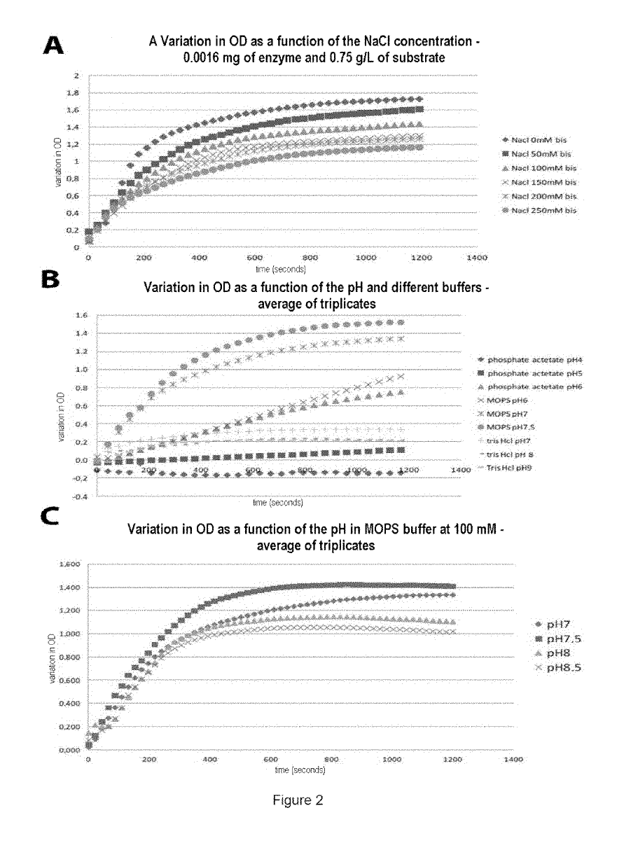

An activity test typically used for the polysaccharide lyases (e.g. [7, 8]) was performed for BN863_22190_cat. It can include in following the absorbance of the reaction medium at 235 nm. In fact, the polysaccharide lyases liberate oligosaccharides exhibiting an unsaturated monosaccharide at the non-reducing end which absorb at 235 nm. This test thus made it possible to determine the optimal salinity of the medium for the enzyme (FIG. 2A), the optimal buffer (FIG. 2B) and the optimal pH with the better buffer (FIG. 2C).

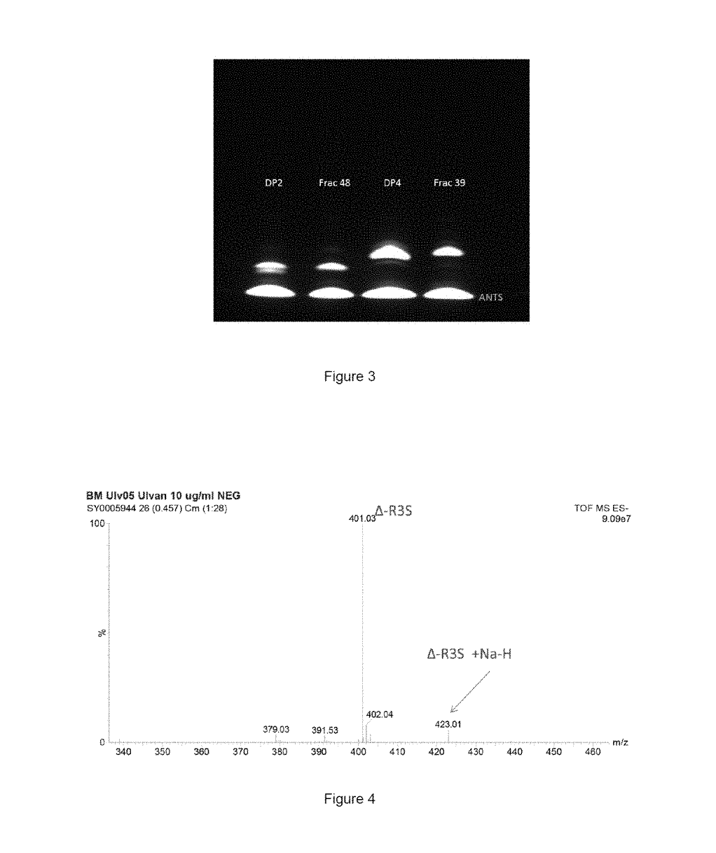

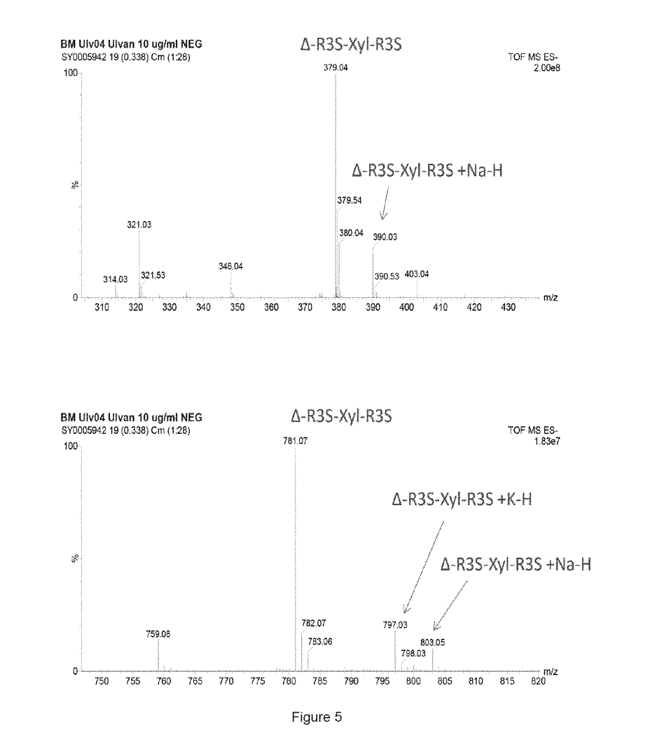

An extensive degradation of a pure ulvan (ULV100, Elicityl, France) was also performed utilizing BN863_22190_cat. The oligo-ulvans liberated were purified by size exclusion chromatography on a system of three interconnected Superdex 30 columns (GE Healthcare, France). These purified oligosaccharides were then analyzed by the technique called FACE (Fluorophore-assisted Carbohydrate Electrophoresis). The reducing end of the oligosaccharides was thus labeled with a fluorescent molecule (8-amino-naphthalene-1,3,6-trisulfonic acid, ANTS) as previously described [9]. The labeled oligosaccharides are then analyzed by polyacrylamide gel electrophoresis. Oligo-ulvans previously obtained in the laboratory by the wild ulvan lyase of N. ulvanivorans PLR [2] were also utilized as standards (DP2: the disaccharide Δ-R3S; DP4: the tetrasaccharide Δ-R3S-Xyl-R3S; the structures of DP2 and DP4 have been determined by NMR [2]). FIG. 3 shows that the terminal oligosaccharides “Fraction 48” and “Fraction 39” migrate at the same size as the standards DP2 and DP4, respectively. The structure of these oligosaccharides generated by BN863_22190_cat. was confirmed by mass spectrometry (BIBS platform, INRA, Nantes) (FIGS. 4 and 5). Consequently, like the wild ulvan lyase of N. ulvanivorans PLR, BN863_22190_cat. cleaves the beta-1,4 bond between the D-glucuronic acid (GlcA)/L-iduronic acid (IduA) residues and the rhamnose-3-sulfate (R3S) endolytically liberating oligosaccharides of different sizes exhibiting an unsaturated sugar A at the non-reducing end. The main end products are the disaccharide Δ-R3S, and the tetrasaccharide Δ-R3S-Xyl-R3. The tetrasaccharides Δ-R3S-GlcA-R3S and A-R3S-IduA-R3S also described in the article Nyvall Collen et al., JBC, 2011 are not observed in the experiment owing to the extensive degradation of the ulvan by BN863_22190_cat. In fact, these two types of tetrasaccharide are cleaved in two by BN863_22190_cat, liberating Δ-R3S disaccharides (FIG. 3). In contrast, the tetrasaccharide Δ-R3S-Xyl-R3S is an end product resistant to the enzymatic cleavage (FIG. 3), owing to the presence of a xylose residue at position 3 from the non-reducing end.

The results show a strong ulvan degradation activity by BN863_22190-cat, confirmed by measurement of the absorbance at 235 nm and by the electrophoresis analysis and by mass spectrometry of the purified oligo-ulvans. By comparison, a residual activity was detected on following the degradation of the ulvan by the ulvan lyase of N. ulvanivorans by spectrophotometry at 240 nm (data not shown).

### REFERENCES

1. Lahaye and Robic, Biomacromolecules, 8: 1765-1774, 2007
2. Nyvall Cohen et al., J. Biol. Chem., 286(49): 42063-42071, 2011
3. International application WO 2011/157966
4. Mann et al., Appl. Env. Microbiol., 79: 6813-6822, 2013
5. Nedashkovskaya, et al (2006). Formosa agariphila sp. nov., a budding bacterium of the family Flavobacteriaceae isolated from marine environments, and emended description of the genus Formosa. Int. J. Syst. Evol. Microbiol. 56: 161-167.
6. Groisillier et al., Microb Cell Fact. 9:45, 2010
7. Michel et al., J. Biol. Chem., 31: 32882-32896, 2004
8. Thomas et al., J. Biol. Chem., 32: 23021-23037, 2013
9. Jackson, Biochem. J., 270: 705-713, 1990.
10. Petersen et al., Nature Methods, 8: 785-786, 2011

1. An ulvan lyase, comprising:
a sequence SEQ ID No: 2, 4 or 5, or a sequence having at least 53% sequence identity with the sequence SEQ ID No: 2 or 4; and
at its N-terminal end, a signal sequence of sequence SEQ ID No: 6.

2. A nucleic acid coding for the ulvan lyase as defined in claim 1.

3. The nucleic acid as claimed in claim 2, further comprising a sequence SEQ ID No: 1 or 3.

4. A vector, comprising:
the nucleic acid as claimed in claim 2.

5. A host cell, comprising:
the nucleic acid sequence as claimed in claim 2.

6. A process for production of the ulvan lyase as defined in claim 1 by genetic recombination utilizing a nucleic acid coding for the ulvan lyase.

7. A process for degradation of ulvans, comprising:
a stage of contacting ulvans with the ulvan lyase as claimed in claim 1, under conditions enabling the degradation of the ulvans by enzymatic digestion by the ulvan.

8. A method of using the ulvan lyase as defined in claim 1 for the production of oligo-ulvans.

9. A vector, comprising:
the nucleic acid as claimed in claim 3.

10. A host cell, comprising:
the nucleic acid sequence as claimed in claim 3.

11. A host cell, comprising:
the vector as claimed in claim 4.

12. A process for production of the ulvan lyase of claim 1 by genetic recombination utilizing a nucleic acid coding for a ulvan lyase that includes a sequence SEQ ID No: 1 or 3.

13. A process for production of the ulvan lyase of claim 1 by genetic recombination utilizing a vector that includes a nucleic acid coding for the ulvan lyase.

14. A process for degradation of ulvans, comprising:
a stage of contacting ulvans with the host cell as claimed in claim 5, under conditions enabling the degradation of the ulvans by enzymatic digestion by the host cell.
